(19) United States
(12) Patent Application Publication
(10) Pub. No.: US 2022/0028552 A1
(43) Pub. Date: Jan. 27, 2022

(54) A METHOD AND DEVICE FOR BUILDING A MODEL FOR PREDICTING EVOLUTION OVER TIME OF A VISION-RELATED PARAMETER

(71) Applicant: ESSILOR INTERNATIONAL, CHARENTON-PONT (FR)

(72) Inventors: Bjorn DROBE, SINGAPORE (SG); Aurélie LE CAIN, CHARENTON-LE-PONT (FR); Yee Ling WONG, SINGAPORE (SG)

(21) Appl. No.: 17/414,198

(22) PCT Filed: Dec. 4, 2019
(86) PCT No.: PCT/EP2019/083723

§ 371 Date: Jun. 15, 2021

(30) Foreign Application Priority Data
Dec. 21, 2018 (EP) 18306805.5

(51) Int. Cl. G16H 50/20; G16H 50/70; G16H 10/60; G16H 50/50; G16H 50/30; A61B 3/00

(52) U.S. Cl. CPC G16H 50/20; G16H 50/70; A61B 3/0025; G16H 50/50; G16H 50/30; G16H 10/60

(57) ABSTRACT

This method for building a prediction model for predicting evolution over time of at least one vision-related parameter of at least one person includes: obtaining successive values respectively corresponding to repeated measurements over time of at least one parameter of a first predetermined type for at least one member of a group of individuals; obtaining evolution over time of the vision-related parameter(s) for the member(s) of the group of individuals; building by at least one processor the prediction model, including associating at least part of the successive values with the obtained evolution over time of the vision-related parameter(s) for the member(s) of the group of individuals, the associating including jointly processing the at least part of the successive values associated with a same one of the parameter(s) of the first predetermined type. The prediction model depends differentially on each of the jointly processed values.

### FIELD OF THE INVENTION

The present invention relates to a method and device for building a prediction model for predicting evolution over time of at least one vision-related parameter of at least one person.

### BACKGROUND OF THE INVENTION

While some factors influencing human vision, such as genetic factors, cannot be modified by the person concerned, some other factors, such as lifestyle, behavior and/or environmental factors, can be modified by everyone. For example, the amount of time spent outdoors, the amount of time spent on work involving near vision, or nutrition may impact vision, by causing for example myopia onset, progression or reduction.

Wearable devices are known that can correct for example a person's reading and/or writing posture and that can collect myopia-related parameters.

However, known devices are often standardized and are thus identical for all persons, i.e. they assume that all persons have similar risks e.g. of myopia onset and progression, which is actually not the case.

In addition, for many existing devices, the predicted myopia progression profile is calculated once and is not updated later on.

Therefore, should the person's lifestyle, behavior and/or environment change after the predicted profile for that person has been calculated, the unchanged predicted profile will become inconsistent and erroneous.

Thus, there is a need for taking into account changes regarding modifiable parameters impacting vision of the person, when building a model for predicting evolution over time of one or more vision-related parameters for that person.

### SUMMARY OF THE INVENTION

An object of the invention is to overcome the above-mentioned drawbacks of the prior art.

To that end, the invention provides a method for building a prediction model for predicting evolution over time of at least one vision-related parameter of at least one person, remarkable in that it comprises:

obtaining successive values respectively corresponding to repeated measurements over time of at least one parameter of a first predetermined type for at least one member of a group of individuals;

obtaining evolution over time of the at least one vision-related parameter for the at least one member of the group of individuals;

building by at least one processor the prediction model, including associating at least part of the successive values with the obtained evolution over time of the at least one vision-related parameter for the at least one member of the group of individuals, such associating including jointly processing the at least part of the successive values associated with a same one of the at least one parameter of the first predetermined type;

the prediction model depending differentially on each of the jointly processed values.

Therefore, the prediction model is built by collecting data from a group of individuals i.e. a whole panel of individuals and by taking into account the possible modification over time of the parameters measured for those individuals. Having the prediction model depend differentially on each of those jointly processed values, i.e. taking into account both those successive values themselves and the results of jointly processing successive values of parameters, makes it possible to obtain a very accurate and consistent dynamic prediction model. Namely, interchanging inputs corresponding to those successive jointly processed values, e.g. by swapping values obtained at different day hours, may have an effect on the built prediction model.

The enhanced prediction capacity potentially offered by the above method for building a prediction model can notably be due to a time-dependent personal vision sensibility of the considered person(s), which is a particular expression of a personal chronotype.

Generally, the chronotype is an attribute of human beings, reflecting at what time of the day their physical functions (hormone level, body temperature, cognitive faculties, eating and sleeping) are active, change or reach a certain level. It is considered as an important predictor of sleep timings, sleep stability, sleep duration, sleep need, sleep quality, morning sleepiness, adaptability to shift work.

The enhanced prediction capacity can, alternatively or further, notably be due to the implicit consideration of time-dependent environment parameters that are not explicitly entered as inputs, but are depending on the times at which the successive values are obtained. Those may notably include light spectral distributions, light ray orientations, light radiance and/or light coherence and/or diffusion properties, whether associated with a natural lighting, an artificial lighting or both together.

Furthermore, the fact that the prediction model depends differentially on each of the jointly processed values makes it possible to identify and/or have better knowledge of parameters that influence the prediction model without being explicitly entered. Chronobiology (in relationship with the recording of sleeping cycles and their characteristics) and light ray orientations can be examples of such parameters.

The invention also provides a device for building a prediction model for predicting evolution over time of at least one vision-related parameter of at least one person, remarkable in that it comprises:

at least one input adapted to receive successive values respectively corresponding to repeated measurements over time of at least one parameter of a first predetermined type for at least one member of a group of individuals and evolution over time of the at least one vision-related parameter for the at least one member of the group of individuals;

at least one processor configured for building the prediction model, including associating at least part of the successive values with the obtained evolution over time of the at least one vision-related parameter for the at least one member of the group of individuals, including jointly processing the at least part of the successive values associated with a same one of the at least one parameter of the first predetermined type;

the prediction model depending differentially on each of the jointly processed values.

The invention further provides a computer program product for building a prediction model for predicting evolution over time of at least one vision-related parameter of at least one person, remarkable in that it comprises one or more sequences of instructions that are accessible to a processor and that, when executed by the processor, cause the processor to:

build the prediction model, including to associate at least part of successive values respectively corresponding to repeated measurements over time of at least one parameter of a first predetermined type for at least one member of a group of individuals with evolution over time of the at least one vision-related parameter for the at least one member of the group of individuals, including to jointly process the at least part of the successive values associated with a same one of the at least one parameter of the first predetermined type;

the prediction model depending differentially on each of the jointly processed values.

The invention further provides a non-transitory computer-readable storage medium remarkable in that it stores one or more sequences of instructions that are accessible to a processor and that, when executed by the processor, cause the processor to:

build a prediction model, including to associate at least part of successive values respectively corresponding to repeated measurements over time of at least one parameter of a first predetermined type for at least one member of a group of individuals with evolution over time of at least one vision-related parameter for the at least one member of the group of individuals, including to jointly process the at least part of the successive values associated with a same one of the at least one parameter of the first predetermined type;

the prediction model depending differentially on each of the jointly processed values.

As the advantages of the device, of the computer program product and of the computer-readable storage medium are similar to those of the method, they are not repeated here.

The device for building a prediction model, the computer program and the computer-readable storage medium are advantageously configured for executing the method for building a prediction model in any of its execution modes.

### BRIEF DESCRIPTION OF THE DRAWINGS

For a more complete understanding of the description provided herein and the advantages thereof, reference is now made to the brief descriptions below, taken in connection with the accompanying drawings and detailed description, wherein like reference numerals represent like parts.

FIG. 1 is a flowchart showing steps of a method for building a prediction model according to the invention, in a particular embodiment.

FIG. 2 is a graph showing a myopia evolution risk profile based on a prediction model obtained by a method for building a prediction model according to the invention, in a particular embodiment.

FIG. 3 is a flowchart showing steps of a predicting method resulting from the use of a prediction model built according to the invention, in a particular embodiment.

FIG. 4 is the graph of FIG. 2 showing in addition a monitoring indicator.

FIG. 5 is a set of two graphs showing examples of multiple risk profiles including predicted evolutions over time obtained by implementing a predicting method resulting from the use of a prediction model built according to the invention, in a particular embodiment.

FIG. 6 is a graph showing two myopia onset risk profiles based on a prediction model obtained by a method for building a prediction model according to the invention, in a particular embodiment.

### DETAILED DESCRIPTION OF THE INVENTION

In the description which follows, the drawing figures are not necessarily to scale and certain features may be shown in generalized or schematic form in the interest of clarity and conciseness or for informational purposes. In addition, although making and using various embodiments are discussed in detail below, it should be appreciated that as described herein are provided many inventive concepts that may embodied in a wide variety of contexts. Embodiments discussed herein are merely representative and do not limit the scope of the invention. It will also be obvious to one skilled in the art that all the technical features that are defined relative to a process can be transposed, individually or in combination, to a device and conversely, all the technical features relative to a device can be transposed, individually or in combination, to a process.

The terms “comprise” (and any grammatical variation thereof, such as “comprises” and “comprising”), “have” (and any grammatical variation thereof, such as “has” and “having”), “contain” (and any grammatical variation thereof, such as “contains” and “containing”), and “include” (and any grammatical variation thereof such as “includes” and “including”) are open-ended linking verbs. They are used to specify the presence of stated features, integers, steps or components or groups thereof, but do not preclude the presence or addition of one or more other features, integers, steps or components or groups thereof. As a result, a method, or a step in a method, that “comprises”, “has”, “contains”, or “includes” one or more steps or elements possesses those one or more steps or elements, but is not limited to possessing only those one or more steps or elements.

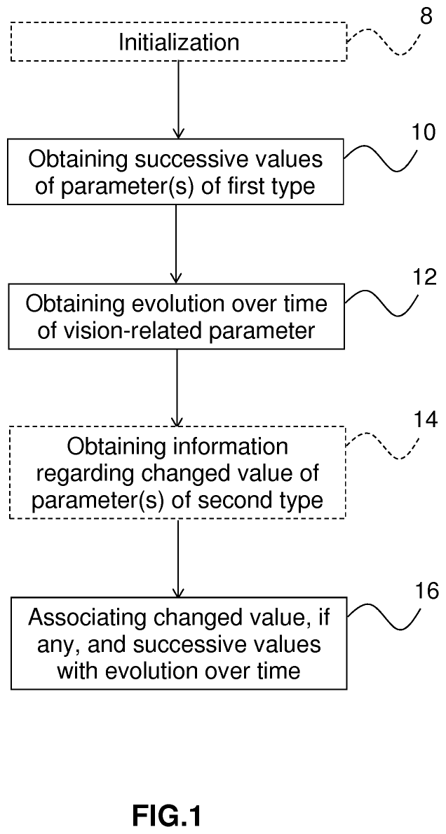

As shown in FIG. 1, a method for building a prediction model for predicting evolution over time of at least one vision-related parameter of at least one person comprises a step 10 of obtaining successive values respectively corresponding to repeated measurements over time of at least one parameter of a first predetermined type for at least one member of a group of individuals.

By way of non-limiting example, the vision-related parameter considered may be the myopia level of the person, which may be expressed in diopters for the left and/or right eye. It may be any other parameter relating to the visual aptitudes or to any visual deficiency of the person, such as hypermetropia, astigmatism, presbyopia, or to any visual disease, such as ocular diseases that can result in visual issues including myopic macular degeneration, retinal detachment and glaucoma. Besides refractive error (expressed in diopters), ocular biometry measurements, such as axial length (in mm), vitreous chamber depth (in mm), choroidal thickness (expressed in μm) and corneal characteristics are other examples of vision-related parameters.

The group of individuals may include any number of individuals who may have either no characteristic in common with each other, or one or more common characteristics, such as, by way of non-limiting examples, their gender and/or date of birth and/or country of birth and/or previous family history and/or ethnic group.

In any case, such fixed parameters of at least one member of the group of individuals may be input into the prediction model either in a preliminary step 8 of initialization, or later on, at any stage of the method. Such input of fixed parameters is optional. The fixed parameters may be available individually for members of the group of individuals, or may be available collectively for subgroups of the group of individuals.

The above-mentioned successive values are not necessarily consecutive in time.

The first type of parameters considered relates for example to the lifestyle or activity or behavior of the individual or person considered.

By way of non-limiting example, parameters of the first type may include a time duration spent outdoors or indoors, a distance between eyes and a text being read or written, a reading or writing time duration, a light intensity or spectrum, duration of sleeping cycles or a frequency or time duration of wearing visual equipment.

More generally, parameters of the first type are any parameters that are likely to influence evolution of the chosen vision-related parameter and that can be measured repeatedly at different time instants.

The measurements may be taken, possibly together with a timestamp, by means of various kinds of sensors adapted to detect the parameter(s) considered.

For instance, light sensors, which may be included in smart eyewear equipment or in a smartphone, may be used to measure intensity or spectrum of environment light. An inertial motion unit (IMU) located for instance in a head accessory may be used to detect posture. An IMU may also be used for measuring the time spent carrying out an outdoor activity. A GPS may be used to detect an outdoor activity or whether the individual is in a rural or in an urban environment. A camera or a frame sensor may be used to detect the frequency and/or time duration of wearing eyeglasses. A memory may be used for registering the date of current visual equipment, given the fact that old visual equipment may influence visual aptitudes.

After step 10, a step 12 of obtaining evolution over time of the chosen vision-related parameter(s) is performed for the same individuals of the group of individuals for whom the successive values have been obtained.

Such evolution over time may be obtained by repeatedly measuring over time the chosen vision-related parameter(s) for those individuals and/or by collecting information relating to the values of the vision-related parameter(s) provided by the individual, through any appropriate interface, to a processor building the prediction model.

The measurement frequencies may differ for the various parameters measured at step 10 and they may have no relationship with the measurement frequencies of step 12.

For example, parameters of the first type may be measured at least once a day. As a variant, using a smart frame, parameters of the first type may be measured at a frequency higher than 1 Hz.

Next, as an optional feature, an additional step 14 may be performed, of obtaining information regarding a changed value of one or more parameters of a second predetermined type for at least one individual among those individuals for whom the successive values have been obtained.

The parameters of the second type are any punctual or occasional events that are likely to influence evolution of the chosen vision-related parameter and that can be obtained at least once.

By way of non-limiting example, parameters of the second type may be a move from an urban area to a countryside area, change of correction type, change of power of corrective lenses, or becoming pregnant.

During the following step 16, performed by at least one processor, at least part of the successive values obtained at step 10 are associated with the evolution over time obtained at step 12. Such part of the successive values is a selected series of values, taken among the values obtained previously. The selected values are not necessarily consecutive in time. In a particular embodiment, the selected series comprises at least three successive values.

In addition, at least part of the fixed parameters mentioned previously may also be taken into account in the associating process.

If the optional step 14 is omitted, the associating performed at step 16 includes jointly processing the above-mentioned part of the successive values obtained for the same parameter of the first type. By way of non-limiting example, such joint processing may include calculating an average value and/or a standard deviation value, over a predetermined period of time, of a given number of successive values of the same parameter of the first type. It may also include an aggregation of successive values over a predetermined period of time and such aggregation may then also be averaged over a predetermined period of time.

If the optional step 14 is carried out, the associating performed at step 16 includes associating with the obtained evolution over time of the chosen vision-related parameter the changed value of the parameter of the second type together with the above-mentioned part of the successive values.

Thus, whether the optional step 14 is carried out or not, a correlation table or any other database means can be built and stored in a non-transitory computer-readable storage medium such as a read-only memory (ROM) and/or a random access memory (RAM), in which obtained values of parameters correspond to a determined evolution over time of the chosen vision-related parameter.

According to the disclosure, in addition to the jointly processed values, the correlation table or other database means takes into account each of those individually obtained successive values, or at least some of them, i.e. at least two and preferably at least three. In other words, the prediction model will differ as a function of each of those successive values, i.e. the prediction model depends differentially on each of those jointly processed values and not only on the results of the joint processing.

The prediction model may depend differentially on each of the jointly processed values through joint processing. For example, an average may rely on distinctive weights associated with respectively different successive values, e.g. a higher weight at 12 hours PM than at 9 hours PM. In alternative implementations, which can be combined with the previous ones, the joint processing and the differential consideration of successive values are effected separately. For example, an aggregation of successive values forms one prediction input and several of those values form additional prediction inputs.

The order in which steps 8, 10, 12, 14 and 16 have been described is a non-limiting example. They may be carried out in any other order. For example, the associating step 16 may be started as soon as part of the successive values and part of the evolution over time of the vision-related parameter(s) have been obtained and steps 10, 12 and 14 may be carried out at the same time as step 16 continues.

The prediction model building method may be implemented in a server.

A prediction model building device according to the invention comprises at least one input adapted to receive the successive values for at least one member of the group of individuals as described above, as well as evolution over time of the considered vision-related parameter(s) for such member(s) of the group of individuals. The device also comprises at least one processor configured for building the prediction model as described above.

Such a device may comprise a display unit and/or a smartphone or smart tablet or smart eyewear, in addition to a server, if the method is implemented in a remote centralized fashion in the server.

In a particular embodiment of the prediction model building method, the group of individuals may also include the person for whom the evolution over time of one or more vision-related parameters is to be predicted by the prediction model built according to the building method described in the present application. In other words, steps 10, 12, 16 and possibly step 14 are also performed for that person.

In a particular embodiment, the processor used at step 16 may implement a machine learning algorithm. Namely, one or more neural networks may be trained by inputting series of successive values for numerous individuals and building a correlation table or any other database means containing lots of data, for better accuracy of the prediction model.

In such an embodiment, the associating of step 16 may be implemented by assigning weights to node connections in the neural network.

Self-reported parameters provided by the individuals of the group may also be taken into account by the prediction model building.

By way of non-limiting example, self-reported parameters may be input in the machine learning algorithm, such as, by way of non-limiting examples, their respective genders, ethnic group, number of myopic parents, school marks, results of intellectual quotient tests, data from social networks, refraction values of their visual equipment, or a genetic risk score related to a visual deficiency or disease. Such self-reported parameters will in turn modify the prediction model. Other fixed parameters as well as parameters of the first and/or second type may also be self-reported, as well as the evolution over time of the vision-related parameter(s) of the individuals of the group.

For inputting self-reported parameters or parameters of the second type, the device for building the prediction model may include display means and/or the smartphone or smart tablet already used for taking first type parameter measurements, or any other kind of user interface, including audio interfaces.

The prediction model built by the method previously described may be exploited in a large number of ways, in order to provide the person with information regarding the predicted evolution over time of one or more vision-related parameters of that person.

If the chosen vision-related parameter is for example the onset or progression risk of a given visual deficiency, the predicted model may be used to illustrate the evolution over time of that risk of in the form of a profile graph.

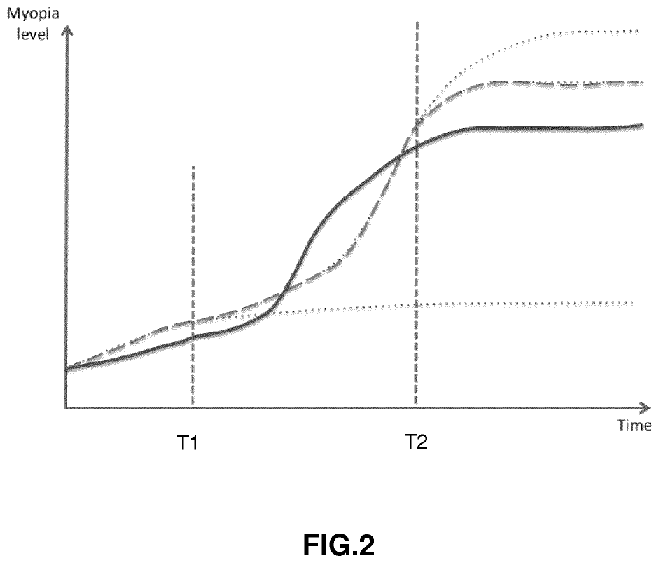

FIGS. 2 and 6 show such graphs in an example where the visual deficiency is myopia.

In FIG. 2, the myopia level evolution of a monitored person is represented as a function of time.

In FIG. 6, the myopia onset risk is represented as a function of time.

In FIG. 2, the unbroken curve shows the actual measured myopia evolution profile. The dashed curve shows the predicted myopia risk profile that updates as a function of the modification of the dynamic predicted evolution. The dotted curves show the myopia risk profiles predicted before inputting modified values of input parameters.

As a parameter of the first type, time spent on work involving near vision is measured. From time T1, with an increase in time spent on such work, the risk of myopia progression increases, which is reflected by a sharp rise in the predicted myopia risk profile (dotted curves). At time T2, the monitored person moves from a city to the countryside. This is reflected by a gradual plateau in the predicted myopia risk profile.

It can be seen that the predicted profile substantially corresponds to the actual measured evolution profile, contrary to the predicted profiles not updated for taking into account parameter modifications from T1 and at T2.

In FIG. 6, at an original time, two scenarios are considered in predicting the myopia onset risk. In a first scenario, the monitored person continues to live in a city while keeping near vision screen work habits, which leads to myopia triggering at a future time T3, followed by a relatively sharp increase of the predicted myopia level over time. In a second scenario, the monitored person moves to live in the countryside and adopts modified habits with less near vision screen work, which leads to myopia triggering at a future time T4 greater than T3, and to a slightly lower myopia evolution. The lower myopia evolution risk in the second scenario compared with the first scenario is thereby quantified.

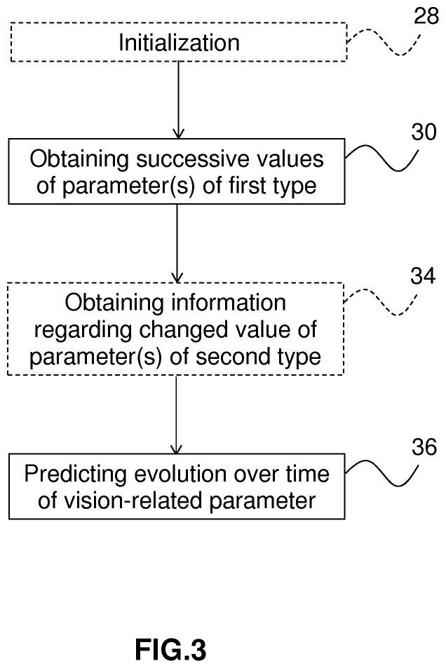

More generally, as shown in FIG. 3, the proposed prediction model building method may be used in a method for predicting evolution over time of at least one vision-related parameter of at least one person. The predicting method comprises a step 30 of obtaining successive values, for the person, respectively corresponding to repeated measurements over time of at least one parameter of the first type and a step 36 of predicting by at least one processor the evolution over time of the vision-related parameter of the person from the successive values obtained at step 30, by using the previously described prediction model associated with the group of individuals.

Step 30 is performed for the person in a similar manner as step 10 for the individuals of the group.

Similarly to the optional initialization step 8 in FIG. 1, an optional initialization step 28 may collect fixed parameters for the person such as gender and/or date of birth and/or country of birth and/or family history and/or ethnic group. Step 28 may be carried out either in a preliminary step of initialization, or later on, at any stage of the predicting method.

In a particular implementation, before the predicting step 36, an optional step 34 may be performed, of obtaining information regarding a changed value of at least one parameter of the second type for the person.

The predicting step 36 uses the prediction model.

If the optional step 34 is omitted, the predicting step 36 includes associating at least part of the successive values for the person with the predicted evolution over time of the chosen vision-related parameter of the person. The associating operation includes jointly processing the above-mentioned part of the successive values associated with a same parameter of the first type.

Such part of the successive values is a selected series of values, taken among the values obtained previously. The selected values are not necessarily consecutive in time. In a particular implementation, the selected series comprises at least three successive values.

If the optional step 34 is performed, the predicting step 36 further includes associating with the predicted evolution over time of the chosen vision-related parameter for the person the changed value of the parameter(s) of the second type together with the above-mentioned part of the successive values of the parameter(s) of the first type for the person.

According to the disclosure, whether the optional step 34 is performed or not, as for the prediction model, the predicted evolution takes into account, not only the results of the joint processing of those successive values or at least some of them, i.e. at least two and preferably at least three, but also each of those successive values, or at least some of them, so that the predicted evolution will differ as a function of each of those successive values, i.e. the prediction model depends differentially on each of those jointly processed values.

A predicting device according to the disclosure comprises at least one input adapted to receive the successive values for at least one person as described above. The device also comprises at least one processor configured for predicting the evolution over time of the considered vision-related parameter of the person as described above.

Such a device may comprise a display unit and/or a smartphone or smart tablet or smart eyewear, which may be the same as the display unit and/or smartphone or smart tablet or smart eyewear or server comprised in the prediction model building device. In case the predicting method is implemented in a remote centralized fashion in a server, outputs from the server are communicated to the user through a communication network, possibly through wireless or cellular communication links.

In a particular implementation of the predicting method, the group of individuals with whom the prediction model is associated may also include the person for whom the evolution over time of one or more vision-related parameters is to be predicted by the prediction model built according to the building method described in the present application. In other words, steps 10, 12, 16 and possibly step 14 are also performed for that person.

In case self-reported parameters are provided by the individuals of the group, the same self-reported parameters for the person may also be input into the prediction model, such as the person's gender, ethnicity, number of myopic parents, school marks, results of intellectual quotient tests, data from social networks, refraction values of visual equipment, or a genetic risk score related to a visual deficiency or disease.

Other advantageous aspects of the predicting method relate to a large number of possibilities of interacting with the person, in particular by providing feedback to the person (and/or to other people such as for example the person's parents, if the person is a child) regarding the predicted evolution over time of the at least one vision-related parameter of that person.

As a first possibility of interacting with the person, the predicted evolution over time of the chosen vision-related parameter(s) of the person may be made available in the form of graphs of the type illustrated in FIG. 2, which may be visualized for example on the screen of a smartphone or smart tablet, through a mobile application.

As another possibility of interacting with the person, the predicting method may comprise triggering the sending of one or more alert messages to the person, on the basis of the predicted evolution over time of the considered vision-related parameter of the person. In this respect, the contents and/or frequency of the alert message(s) may vary according to a level of risk related to the considered vision-related parameter of the person.

For example, if the considered vision-related parameter of the person is the risk of myopia onset or progression, a person having a high myopia risk will be alerted that he/she is reading too close at a trigger threshold of less than 30 cm, whereas a person having a low myopia risk will be alerted at a trigger threshold of less than 20 cm.

Such a trigger threshold may vary over time for a given person, depending on the evolution over time of the predicted myopia risk for that person.

The frequency of the alert message(s) may vary similarly.

An alert message may for example timely prompt, encourage or remind the person to take or maintain healthy eye-using habits, which will help preserve the person's visual aptitudes. Therefore, persons can change their behavior from such timely reminders or prompts. A very simple visualization allows the persons to know if their behavior if beneficial or harmful for eye health.

If the considered vision-related parameter is the level of myopia, the reminders or prompts will discourage activities that confer a risk of myopia onset or progression and/or will encourage activities that have a protective effect against myopia onset or progression.

The table below gives examples of activities and of corresponding actions implemented by a smartphone or smart tablet included in a predicting device according to the invention, in the myopia example.

| | General trigger | Action/nudge |
|---|---|---|
| Activity | threshold | from the device |
| Near vision | Near vision distance | Vibration of device |
| work (e.g. | falls below 30 cm | and/or audio reminders |
| reading or | for more than 5 min | and/or visual prompts |
| writing) | or time spent on | from mobile application |
| | near vision work | |
| | exceeds 45 min | |
| Outdoor | Outdoor | Prompts that interact |
| time | (luminance > 1000 | with persons and |
| | lux) time exceeds | encourage them to |
| | 20 min | prolong time spent |
| | | outdoor |
| Indoor | Indoor | Prompts that interact |
| time | (luminance < 200 | with persons to nudge |
| | lux) time exceeds | them to go outdoors |
| | 2 h during day time | and/or visual prompts |
| | | from mobile application |

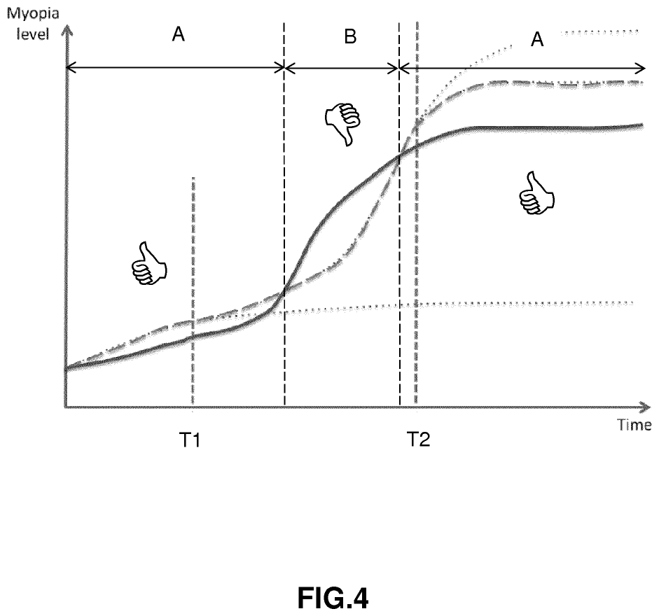

As shown in FIG. 4, as another possibility of interacting with the person, the predicting method may comprise providing the person with a monitoring indicator, having a first state if the predicted evolution over time of the vision-related parameter(s) of the person is less favorable than an actual measured evolution over time of the vision-related parameter(s) of the person, or a second state if the predicted evolution over time of the vision-related parameter(s) of the person is more favorable than the actual measured evolution over time of the vision-related parameter(s) of the person.

Thus, in the graph of FIG. 4, which shows the same curves as in FIG. 2, a monitoring indicator having the form of a hand has the thumb upwards in both areas referred to by “A”, in order to reflect the fact that in those areas, the predicted evolution over time of the myopia level of the person is less favorable than an actual measured evolution over time of that myopia level and it has the thumb downwards in the area referred to by “B”, in order to reflect the fact that in that area, the predicted evolution over time of the myopia level of the person is more favorable than an actual measured evolution over time of that myopia level.

As another possibility of interacting with the person, multiple optimized targets or graphs showing risk profiles can be provided to the person and/or person's parents, based on several scenarios, showing both good and bad eye-using habits, in order to recommend changes in behavior, for example going outdoors to play in case the vision-related parameter is the myopia level or risk, and in order to encourage healthy habits, for example habits that help in preventing myopia onset or in slowing down myopia progression. For instance, the prediction model will calculate and present an ideal myopia risk profile graph, which has been optimized based on the recommended activity, if the person performs the recommended activity, such as going outdoors and spending more time outdoors.

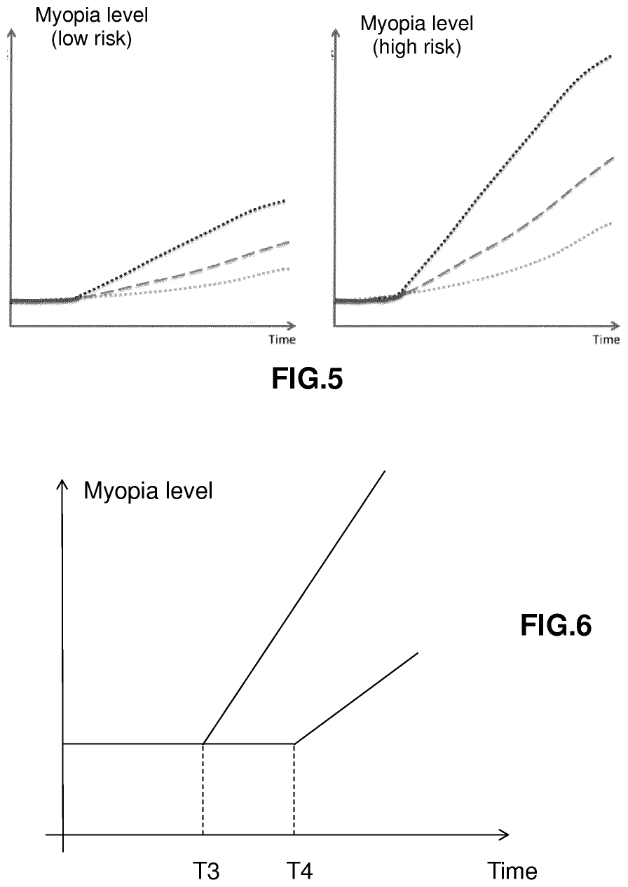

FIG. 5 shows examples of such multiple risk profiles in case the vision-related parameter is the myopia level.

The graph on the left of FIG. 5 shows the evolution over time of the person's myopia level in case the person has a low risk of myopia progression.

The graph on the right of FIG. 5 shows the evolution over time of the person's myopia level in case where the person has a high risk of myopia progression.

On both graphs, the respective unbroken curve portions show the actual measured myopia evolution profiles until a current time, the dashed curves show the predicted myopia risk profiles beyond that current time, which update as a function of the modification of the dynamic prediction model, depending on the changes in the person's eye-using habits and/or behavior. The two dotted curves on each graph show the myopia risk profiles in scenarios where the person would follow or would not follow recommendations for changing eye-using habits and/or behavior. The upper dotted curves correspond to scenarios where the person does not follow recommendations and the lower dotted curves correspond to scenarios where the person follows recommendations.

The dotted curves can be accompanied by the display of an explanation message, for example “If you continue spending too much time on near vision work, the risk of myopia will increase” for the upper dotted curves and “If you go outdoors and play, the risk of myopia will drop” for the lower dotted curves.

As another possibility of interacting with the person, the predicting method may comprise providing the person with a maximal value of a reduction or slowing down of a progression of a visual deficiency of the person, as a function of changes in the value of at least one parameter of the first and/or second predetermined type of the person.

For example, if the person's myopic progression is initially estimated to be around 1 diopter per year, it may be possible for that person to achieve a maximal reduction of myopia progression if the person adopts the most healthy behavior and/or activity and/or environment. For instance, maximal time spent in outdoor activity and a high reading distance may reduce myopia progression to 0.4 diopter per year, so that the maximal reduction of myopia progression would be 0.6 diopter per year. On the contrary, if the person's behavior and/or activity and/or environment is not optimal, it might lead to a reduction of myopia progression of only 0.3 diopter per year, which corresponds to a ratio of 50% with respect to the maximal possible reduction.

Any of the methods described above may be computer-implemented. Namely, a computer program product comprises one or more sequences of instructions that are accessible to a processor and that, when executed by the processor, cause the processor to carry out steps of the method for building a prediction model and/or steps of the method for predicting evolution over time of at least one vision-related parameter as described above.

The prediction model may be used for example remotely in a cloud, or locally in a smart frame. The updating and recalculating of the model may advantageously be performed in the cloud.

The sequence(s) of instructions may be stored in one or several computer-readable storage medium/media, including a predetermined location in a cloud.

For building the prediction model, the processor may receive from the various sensors, for example via wireless or cellular communication links, the successive values respectively corresponding to the repeated measurements over time of the parameter(s) of the first predetermined type for the member(s) of the group of individuals and/or for the person.

Although representative methods and devices have been described in detail herein, those skilled in the art will recognize that various substitutions and modifications may be made without departing from the scope of what is described and defined by the appended claims.

1. A method for building a prediction model for predicting evolution over time of at least one vision-related parameter of at least one person, comprising:
obtaining successive values respectively corresponding to repeated measurements over time of at least one parameter of a first predetermined type for at least one member of a group of individuals;
obtaining evolution over time of said at least one vision-related parameter for said at least one member of said group of individuals;
building by at least one processor said prediction model, including associating at least part of said successive values with said obtained evolution over time of said at least one vision-related parameter for said at least one member of said group of individuals, said associating including jointly processing said at least part of said successive values associated with a same one of said at least one parameter of said first predetermined type;
said prediction model depending differentially on each of the jointly processed values.

2. The method according to claim 1, further comprising, before said building of said prediction model, obtaining information regarding a changed value of at least one parameter of a second predetermined type for said at least one member of said group of individuals, and wherein said building further includes associating said changed value together with said at least part of said successive values, with said obtained evolution over time of said at least one vision-related parameter for said at least one member of said group of individuals.

3. The method according to claim 1, wherein said at least part of said successive values comprises at least three of said successive values.

4. The method according to claim 1, wherein said at least one person belongs to said group of individuals.

5. The method according to claim 1, wherein said at least one parameter of said first predetermined type is a parameter relating to the lifestyle or activity or behavior.

6. The method according to claim 5, wherein said at least one parameter is a time duration spent outdoors or indoors, a distance between eyes and a text being read or written, a reading or writing time duration, a light intensity or spectrum, or a frequency or time duration of wearing visual equipment.

7. The method according to claim 1, wherein said building further takes account of self-reported parameters.

8. The method according to claim 1, wherein said at least one parameter of said first predetermined type is measured at least once a day.

9. The method according to claim 1, wherein said at least one parameter of said first predetermined type is measured at a frequency higher than 1 Hz.

10. The method according to claim 1, wherein said building uses a machine learning algorithm.

11. A device for building a prediction model for predicting evolution over time of at least one vision-related parameter of at least one person, the device comprising:
at least one input adapted to receive successive values respectively corresponding to repeated measurements over time of at least one parameter of a first predetermined type for at least one member of a group of individuals and evolution over time of said at least one vision-related parameter for said at least one member of said group of individuals;
at least one processor configured for building said prediction model, including associating at least part of said successive values with said obtained evolution over time of said at least one vision-related parameter for said at least one member of said group of individuals, including jointly processing said at least part of said successive values associated with a same one of said at least one parameter of said first predetermined type;
said prediction model depending differentially on each of the jointly processed values.

12. The device according to claim 11, further comprising display means and/or a smartphone or smart tablet or smart eyewear.

13. (canceled)

14. A non-transitory computer-readable storage medium, on which is stored one or more sequences of instructions that are accessible to a processor and that, when executed by said processor, cause said processor to:
build a prediction model, including to associate at least part of successive values respectively corresponding to repeated measurements over time of at least one parameter of a first predetermined type for at least one member of a group of individuals with obtained evolution over time of at least one vision-related parameter for said at least one member of said group of individuals, including to jointly process said at least part of said successive values associated with a same one of said at least one parameter of said first predetermined type;
said prediction model depending differentially on each of the jointly processed values.

15. The method according to claim 2, wherein said at least part of said successive values comprises at least three of said successive values.

16. The method according to claim 2, wherein said at least one person belongs to said group of individuals.

17. The method according to claim 3, wherein said at least one person belongs to said group of individuals.

18. The method according to claim 2, wherein said at least one parameter of said first predetermined type is a parameter relating to the lifestyle or activity or behavior.

19. The method according to claim 3, wherein said at least one parameter of said first predetermined type is a parameter relating to the lifestyle or activity or behavior.

20. The method according to claim 4, wherein said at least one parameter of said first predetermined type is a parameter relating to the lifestyle or activity or behavior.

21. The method according to claim 2, wherein said building further takes account of self-reported parameters.
